(19) United States
(12) Patent Application Publication
(10) Pub. No.: US 2001/0046487 A1
(43) Pub. Date: Nov. 29, 2001

(54) Methods for loading platelets, stabilizing platelets for dry storage and compositions obtained thereby

(76) Inventors: Bruce J. Roser, Cambridge (GB); Diana de Vos, Cambridgeshire (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/894,579

(22) Filed: Jun. 28, 2001

Related U.S. Application Data
(63) Continuation of application No. 08/366,810, filed on Dec. 30, 1994, now abandoned.

(51) Int. Cl. A61K 45/00; A61K 31/715; C12N 5/08

(52) U.S. Cl. 424/93.7; 435/372; 514/53

(57) ABSTRACT

The invention provides methods for drying platelets to obtain compositions which are storage stable over a wide range of temperatures and for an extended period of time. The invention also provides methods for permeabilizing platelets which allows them to be loaded with various compounds.

### TECHNICAL FIELD

This invention relates to methods of loading platelets for drug delivery and/or subsequent stabilization. More specifically, it relates to a process of permeabilizing platelets by acid treatment to introduce stabilizing agents and drying.

### BACKGROUND ART

Blood platelets are one of the complex components involved in maintaining hemostasis. When endothelium is damaged, platelets adhere to exposed surfaces composed of collagen, microfibrils, and basement membrane. Once adhered, platelets release granules that promote hemostatic mechanisms and functions resulting in vasoconstriction and recruitment of other platelets to form an aggregated mass called a platelet plug. The result is to activate coagulation proteins which provide the network to stabilize the platelet plug and reduce bleeding, allowing tissue repairs to occur.

Platelets are transfused to patients for many clinical indications. For instance, platelet infusions are used to correct deficiencies or dysfunctions of circulating platelets as a result of trauma, disease, or drug induced dysfunction.

A major difficulty in using isolated platelets is their short shelf-life. Platelets are only approved by the FDA for storage in a liquid state for up to five days at room temperature, during which time the functional properties rapidly deteriorate.

Further drawbacks of storing platelets in a liquid state include the necessity of considerable storage space and constant agitation within bags of specially developed gas permeable plastics. Typically, platelets are stored in a suspending plasma volume of 45 to 65 ml. Recently, a study reported liquid storage establishing a minimum plasma volume of 30-50 ml. Home et al. (1994) Transfusion 34:39. This storage method still requires considerable space, however, and the shelf life is not extended beyond approximately five days. Another study reported a method for storing platelets in artificial medium containing cyclic 3′-5′ adenosine monophosphate. Bode et al. (1994) Blood 83:1235. However, this method requires liquid storage and the storage time is prolonged to only two weeks.

A method of storing platelets by freeze drying has been described. Bateson et al. (1994) Transfusion Med. 4:213. This method also has drawbacks. The electrokinetic properties of human cryopreserved platelets are different from those of fresh matched platelets. The cryopreserved platelets also exhibit significant changes in surface membrane morphology, including disruption of the platelet membrane, as well as loss of sialic acid residues. Platelets are activated during the process of freeze drying and can only be used as a hematology standard.

It would be useful to have a method for storing platelets at ambient temperature for long periods of time. Even more advantageous would be a method for long term storage of dried platelets. Storage of dried platelets would require less space than storage of platelets in liquid due to reduced volume and would not require constant agitation, thus saving storage and transportation costs.

Adenosine 5′-triphosphate (ATP) is a ubiquitous molecule found in all cells. In addition to supplying energy for the execution of cellular functions, ATP is also known to alter several cellular functions and cell membrane properties. ATP induces pore formation that results in membrane permeability to otherwise impermeant soluble molecules. This has been observed in many cell types, mostly of rodent origin, including rat mast cells, certain transformed endothelial cell lines, mast cells, macrophages and lymphocytes (Cockcroft and Gomperts (1979) Nature 279:541; and Tatham et al. (1988) Europ. J. Pharm. 147:13); transformed mouse fibroblasts (Arav and Friedberg (1985) Biochim. Biochys. Acta 820:183); mouse macrophages, mast cells, hepatocytes, mononuclear and polymorphonuclear phagocytes (Steinberg et al. (1987) J. Biol. Chem. 262:8884); mouse lymphocytes (Di Virgilio (1989) J. Immunol. 143:1955); and human epidermal Langerhans cells (dendritic leukocytes) (Girolomoni et al. (1993) J. Invest. Derm. 100:287).

Trehalose, α-D-glucopyranosyl-α-D-glucopyranoside, is a naturally occurring, non-reducing disaccharide which was initially found to be associated with the prevention of desiccation damage in certain plants and animals which can dry out without damage and revive when rehydrated. Trehalose has been shown to be useful in preventing denaturation of proteins, viruses and foodstuffs during desiccation and subsequent storage. See U.S. Pat. Nos. 4,891,319; 5,149,653; 5,026,566; Blakeley et al. (1990) Lancet 336:854; Roser (July 1991) Trends in Food Sci. and Tech., pp. 166-169; Colaco et al. (1992) Biotechnol. Internat., pp. 345-350; Roser (1991) BioPharm. 4:47; Colaco et al. (1992) Bio/Tech. 10:1007; and Roser et al. (May 1993) New Scientist, pp. 25-28.

Trehalose stabilizes the cell membrane under various stressful conditions. Trehalose is linked to the ability of yeast cells to survive complete dehydration. Eleutherio et al. (1993) Biochim Biophys Acta 1156:263. Trehalose is also known to stabilize lyophilized proteins, such as methanol dehydrogenase (Argall and Smith (1993) Biochem. Mol. Biol. Int. 30:491), and to confer thermoprotection to enzymes from yeast. Hottiger et al. (1994) Eur. J. Biochem. 219:187.

The methods described herein provide platelets which can be loaded with drugs and/or stabilizing agents. Platelets loaded with stabilizing agents and dried can be stored indefinitely at ambient temperatures. During storage the functional properties of the platelets are unchanged.

### SUMMARY OF THE INVENTION

The present invention provides methods of permeabilizing platelets. Such permeabilization allows loading of platelets with various compounds and can also be the initial step of stabilizing platelets for storage. The method of stabilizing platelets for storage includes treating platelets with acid and a stabilizing agent, and drying the treated platelets. The invention also includes compositions comprising the platelets loaded with the compounds, dried platelets and platelets reconstituted from the dried preparations.

Accordingly, one aspect of the invention is a method for permeabilizing platelets by treating them with an acid under conditions effective to permeabilize the platelets.

In another aspect of the invention, a method is provided for loading compounds into platelets. The method includes treating isolated platelets with an acid under conditions effective to permeabilize the platelets and incubating the platelets with at least one compound.

In another aspect of this invention, a method is provided for stabilizing platelets by treating platelets with an acid and a stabilizing agent, and drying the platelets.

In another aspect of the invention, compositions of platelets dried according to the invention are provided. The platelets are storage stable for an indefinite period of time and for at least six weeks, and, upon reconstitution, are suitable for use in any indication for which fresh platelets are used.

In another aspect of the invention, reconstituted, dried platelets are provided. The reconstituted platelets are suitable for use in any indication for which fresh platelets are used. Suitable indications for reconstituted dried platelets include, but are not limited to, transfusion.

In another aspect of the invention, a method is provided for delivering therapeutic agents. This method includes treating platelets with an acid, incubating the permeabilized platelets with a stabilizing agent and a therapeutic agent, and administering the platelets.

In another aspect of the invention, a method is provided for producing platelets suitable for purification of platelet factors. This method includes treating platelets with a suitable acid, incubating the platelets with a stabilizing agent, and drying and recovering the platelets.

### BRIEF DESCRIPTION OF THE DRAWINGS

FIGS. 1(a)-(c) show the morphology of platelets in control plasma

(FIG. 1(a)), before

(FIG. 1(b)) and after

(FIG. 1(c)) acid shock permeabilization. The morphology of the platelets is depicted by the Coulter curves.

FIGS. 2(a)-(b) show the ATP release response from platelets before

(FIG. 2(a)) and after

(FIG. 2(b)) acid shock permeabilization. The control (FIG. 2(a)) shows 2.2 nmole release and the sample exposed to acid shock permeabilization (FIG. 2(b)) shows 1.3 nmole release.

FIGS. 3(a)-(b) show the aggregative response of platelets before

(FIG. 3(a)) and after

(FIG. 3(b)) acid shock permeabilization.

FIGS. 4(a)-(g) show morphology of reconstituted platelets before drying

(FIG. 4(a)), after storage of 1 day

(FIG. 4(b)), 7 days

(FIG. 4(c)), before drying

(FIG. 4(d)), after storage of 14 days

(FIG. 4(e)), 21 days

(FIG. 4(f)), and 28 days

(FIG. 4(g)).

FIGS. 5(a) and (b) are photographs of observations by light microscopy of dried, reconstituted platelets

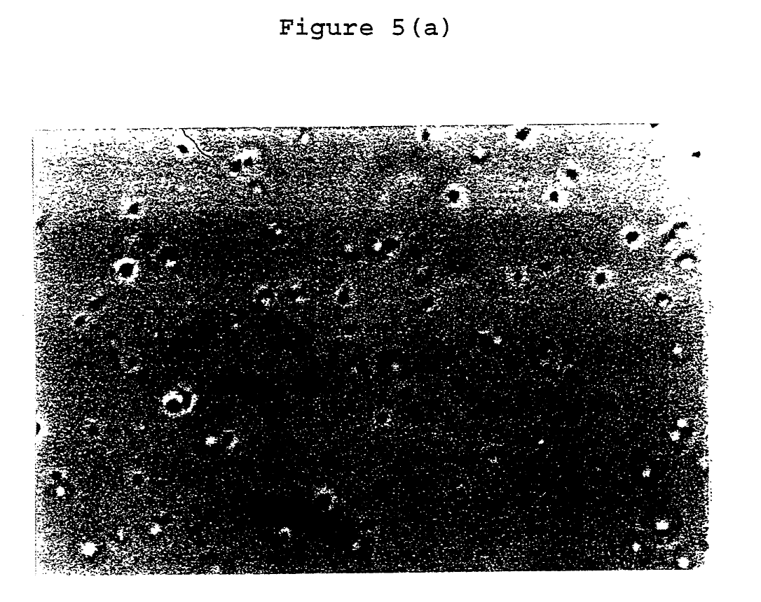

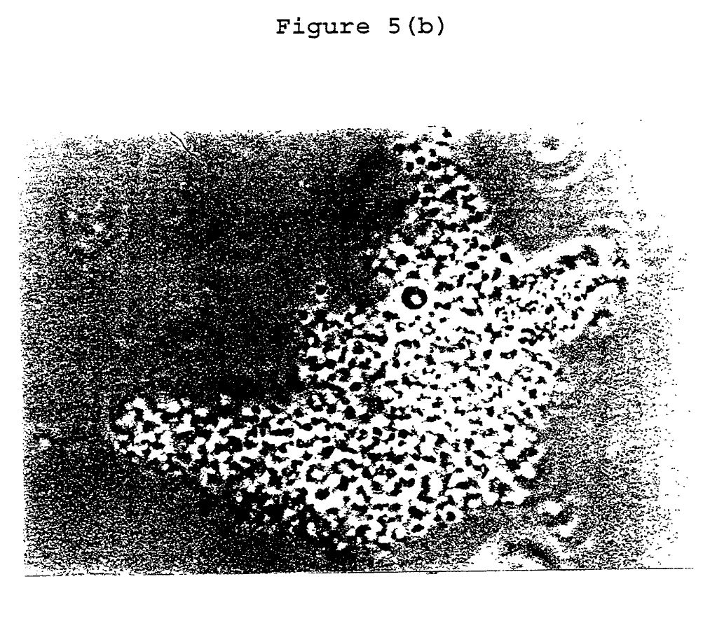

(FIG. 5(a)) and reconstituted platelets aggregated in response to an agonist

(FIG. 5(b)).

### MODES FOR CARRYING OUT THE INVENTION

The invention encompasses methods for permeabilizing platelets which is particularly useful of loading of compounds into platelets. The methods for permeabilizing include treating isolated platelets with an acid in an amount and under conditions effective to permeabilize the platelets. Suitable acids and effective conditions are described below.

The permeabilized platelets can be incubated with at least one compound to be loaded into the platelets. Examples of suitable compounds include, but are not limited to, stabilizing agents, tracers, fluorescent tags and other imaging substances such as radiolabels, cryoprotectants, nucleic acids, and bioactive materials. Bioactive materials particularly suited to incorporation into platelets include, but are not limited to, wound healing factors, cytokines and drugs. Examples of bioactive materials include, but are not limited to, antiinflammatory drugs, analgesics, antiarthritic drugs, antispasmodics, antidepressants, antipsychotics, tranquilizers, antianxiety drugs, narcotic antagonists, antiparkinsonism agents, cholinergic agonists, chemotherapeutic drugs, immunosuppressive agents, antiviral agents, antibiotic agents, appetite suppressants, antiemetics, anticholinergics, antihistaminics, antimigraine agents, coronary, cerebral or peripheral vasodilators, hormonal agents, contraceptives, antithrombotic agents, diuretics, antihypertensive agents, cardiovascular drugs, opioids, and the like.

Suitable bioactive materials also include therapeutic and prophylactic agents. These include, but are not limited to, any therapeutically effective biological modifier. Such modifiers include, but are not limited to, subcellular compositions, cells, bacteria, viruses and molecules including, but not limited to, lipids, organics, proteins and peptides (synthetic and natural), peptide mimetics, hormones (peptide, steroid and corticosteroid), D and L amino acid polymers, oligosaccharides, polysaccharides, nucleotides, oligonucleotides and nucleic acids, including DNA and RNA, protein nucleic acid hybrids, small molecules and physiologically active analogs thereof. Further, the modifiers may be derived from natural sources or made by recombinant or synthetic means and include analogs, agonists and homologs. As used herein, “protein” refers also to peptides and polypeptides. Such proteins include, but are not limited to, enzymes, biopharmaceuticals, growth hormones, growth factors, insulin, monoclonal antibodies, interferons, interleukins and cytokines. Organics include, but are not limited to, pharmaceutically active chemicals with amino, imino and guanidino groups. Suitable steroid hormones include, but are not limited to, estrogen, progesterone, testosterone and physiologically active analogs thereof. Numerous steroid hormone analogs are known in the art and include, but are not limited to, estradiol, SH-135 and tamoxifen. Many steroid hormones such as progesterone, testosterone and analogs thereof are particularly suitable for use in the present invention as they are not absorbed transdermally and, with the exception of a few analogs, are destroyed upon oral administration by the so-called hepatic first pass mechanism. As used herein, “nucleic acids” includes any therapeutically effective nucleic acids known in the art including, but not limited to DNA, RNA and physiologically active analogs thereof. The nucleotides may encode single genes or may be any vector known in the art of recombinant DNA including, but not limited to, plasmids, retroviruses and -adeno-associated viruses.

As discussed in more detail below, the platelets loaded with bioactive materials could themselves be used as a wound healing additive. For example, platelet powder could be applied to wounds or incorporated into wound dressings.

The present invention also provides methods of preparing compositions of dried platelets which are storage stable at ambient temperatures. It has now been found that stabilizing agents can be incorporated into the platelets by acid shock permeabilization.

The methods involve obtaining a suspension of platelets and exposing them to acid shock permeabilization. Platelets may be obtained by any method known in the art. Typically, platelets are obtained by collecting blood into a suitable buffer followed by obtaining platelet rich plasma (PRP) by any method known in the art. After a platelet pellet is obtained from the PRP by centrifugation, the platelet pellet is resuspended in a physiologically acceptable acid solution in an amount and under conditions effective to cause acid shock permeabilization. A variety of acid solutions are suitable for use, including, but not limited to, acid-buffered 2-[N-Morpholino]ethane sulfonic acid (MES), acid-buffered N-[2-Hydroxyethyl]piperazine-N′-[2-ethanesulfonic acid] (HEPES), and acid-buffered piperazine-N,N′-bis[2-ethanesulfonic acid] (PIPES). Preferably, the acid is buffered and in the pH range of 4 to 5. More preferably, the acid is an ATP-buffered HEPES (pH 4.5).

For subsequent stabilization, the acid solution contains at least one stabilizing agent. Preferably, the stabilizing agent is trehalose. In addition to trehalose, suitable stabilizing agents include, but are not limited to, non-reducing glycosides of polyhydroxy compounds selected from sugar alcohols and other straight chain polyalcohols. Other useful stabilizing agents include raffinose, stachyose and melezitose. The sugar alcohol glycosides are preferably monoglycosides, in particular the compounds obtained by reduction of disaccharides such as lactose and maltose. The glycosidic group is preferably a glucoside or a galactoside and the sugar alcohol is preferably sorbitol (glucitol). Particularly preferred compounds are maltitol (4-O-β-D-glucopyranosyl-D-glucitol), lactitol (4-O-β-D-galactopyranosyl-D-glucitol) and palatinit, the sugar alcohol derived from isomaltulose (palatinose), or its two constituent isomers 6-α-D-glucopyranosyl-mannitol and 6-α-D-glucopyranosyl-sorbitol. The acid solution may also contain any of the components discussed herein so as to load the platelets prior to drying.

Preferably, if the platelets are loaded with compounds or stabilizing agents that contain carboxyl and amino, imino or guanidino groups, the platelets are further loaded with at least one physiologically acceptable inhibitor of the Maillard reaction in an amount effective to substantially prevent condensation of amino groups and reactive carbonyl groups in the composition.

The inhibitor of the Maillard reaction can be any known in the art. The inhibitor is present in an amount sufficient to prevent, or substantially prevent, condensation of amino groups and reactive carbonyl groups. Typically, the amino groups are present on the compound and the carbonyl groups are present on the stabilizing agent, or the converse. However, the amino and carbonyl groups may be intramolecular within either the compound or the stabilizing agent. Various classes of compounds are known to exhibit an inhibiting effect on the Maillard reaction and hence to be of use in the compositions are described herein. These inhibitors are generally either competitive or noncompetitive. Competitive inhibitors include, but are not limited to, amino acid residues (both D and L), combinations of amino acid residues and peptides. Particularly preferred are lysine, arginine, histidine and tryptophan. Lysine and arginine are the most effective. There are many known noncompetitive inhibitors known in the art. These include, but are not limited to, aminoguanidine and derivatives and amphotericin B. EP-A-0 433 679 also describes suitable Maillard inhibitors which are 4-hydroxy-5,8-dioxoquinoline derivatives.

Preferably, the stabilizing agent is trehalose in a concentration of from about 0.1 to 20%. Even more preferably, the trehalose concentration is from about 1 to 5%. Preferably, the acid solution contains other functional cofactors and agents including, but not limited to, hirudin, apyrase, and indomethacin. Effective concentrations of these cofactors and agents are amounts sufficient to prevent proteolysis and/or platelet inactivation.

The platelets are exposed to the acid/stabilizing solution for a suitable period of time, under conditions effective to promote permeability of the platelets and loading with the stabilizing agent. Typically, the incubation is for 10-15 minutes at 30-38° C. The incubation step may be from about 5-30 minutes, at a temperature range of from about 25-40° C. Preferably, the incubation is for 10-15 minutes and the temperature range is from about 28-37° C.

The permeabilization is then reversed by increasing the pH of the solution to the physiological range (pH 7.0), by addition of a stop solution. A variety of stop solutions are suitable for use provided that the resultant pH after addition of the stop solution is approximately physiological (i.e., pH 7-7.5). These include, but are not limited to, MES, HEPES, and PIPES. Preferably the stop solution is HEPES-buffered saline.

The platelets are then isolated. Any suitable method may be used to isolate the platelets, including, but not limited to, differential centrifugation. Preferably the treated platelets are centrifuged to form a pellet. The isolated platelets are then resuspended in a buffer containing at least one stabilizing agent. Preferably, the resuspension/drying buffer is a HEPES-buffered saline containing 1% trehalose, 1% bovine serum albumin (BSA), hirudin, apyrase and indomethacin. The trehalose concentration can range from about 0.1% to 20%, and the BSA concentration can range from about 0.1% to 5%.

Alternatively, the platelets can be dried directly without isolation and resuspension if the concentration of trehalose in the final reaction mixture (i.e., after addition of stop solution) is about 0.1% to 20%. Preferably, the trehalose concentration is about 0.5% to 5% and the BSA concentration is about 0.5% to 5%.

The platelets are then dried. Suitable volumes for drying depend on the method of drying used. For instance, during vacuum drying, aliquots of 100 μl to 1 l are preferred. Any method suitable for use in drying biological materials may be used, including, but not limited to, vacuum, air, spray and freeze drying. Preferably, the method used is vacuum drying. During vacuum drying, the vacuum is decreased in a stepwise fashion, keeping the sample temperature above 20° C. Secondary drying is at 30° C. for 16 hours.

The dried platelets can be stored at ambient temperature for extended periods of time. Dried platelets are storage stable for at least six weeks.

The dried platelets can be reconstituted by resuspension in a physiologically acceptable buffer. The buffer can be any buffer of suitable pH containing a substance or substances that exhibit high colloidal osmotic pressure, including, but not limited to, polyethylene glycol (PEG) and hydroxy-ethyl starch (HES). Preferably, the buffer is 20% human serum albumin (HSA) in saline. Reconstituted platelets can be stored for up to 28 days.

The invention further encompasses compositions comprising the platelets obtained by the methods described herein. The compositions include, but are not limited to, permeabilized platelets; permeabilized platelets loaded with at least one compound; dried, permeabilized platelets; dried, permeabilized platelets loaded with at least one compound; reconstituted dried platelets; and reconstituted dried platelets loaded with at least one compound.

The compositions may further comprise any pharmaceutically acceptable vehicle or excipient. For instance, the dried platelets (whether or not loaded with a compound) are particularly suitable for incorporation into topical ointments, creams, gels and salves. Pharmaceutical grade organic carriers and/or diluents suitable for topical use are well known in the art.

The reconstituted platelets are particularly suitable for use in transfusions for both platelet replacement therapy and delivery of bioactive materials. Thus, a particularly preferred composition is a suspension of reconstituted dried platelets (whether or not loaded with a compound) in a buffer suitable for use in intravenous transfusions. The choices of platelet concentration, compound to be delivered and vehicle are well within the skill of one in the art and vary depending on the indication being treated.

The invention further encompasses methods for delivering platelets either alone or as delivery vehicles for therapeutic agents. Therapeutic agents are delivered by first preparing platelets loaded with at least one therapeutic agent, as obtained according to the methods described herein. The platelets are then administered to an-individual. The platelets may be either administered freshly loaded with the compound, or dried and incorporated into a physiologically acceptable vehicle or reconstituted in solution before administration. Therapeutic agents are substances which effect prophylaxis. Examples of suitable therapeutic agents are discussed above. Therapeutic agents prepared by the methods described herein are also encompassed by the invention.

The invention also provides methods for producing platelets suitable for purification of platelet factors. Platelets are treated with a suitable acid, incubated with a stabilizing agent, and then dried and recovered. Platelet factors may then be isolated from the platelets produced thereby. Examples of platelet factors include, but are not limited to, PDGF and vWF. The platelet factors can be isolated by any protein purification techniques known in the art. Suitable methods of protein purification known in the art include, but are not limited to, affinity chromatography, immunoaffinity chromatography, size exclusion chromatography, HPLC and FPLC. Any purification scheme that does not result in substantial degradation of the protein is suitable for use in the present invention.

The following examples are meant to illustrate but not limit the invention.

### EXAMPLE 1

### Acid Shock Permeabilization of Platelets

Platelet rich plasma (PRP) was obtained by collecting 8.3 volumes of blood into 1.7 volumes of ACD (pH 4.5). The platelets were centrifuged in a Damon/1EC Centra 4R centrifuge at 2200 rpm (500 g) for 8 minutes to obtain the PRP (approximately 10⁶ platelets/ml). ACD is made by mixing 2.5 g trisodium citrate; 1.4 g citric acid; 2.0 g dextrose; and deionized water to a volume of 100 ml. The buffer is filtered through a 0.2 μm sterile filter and stored at 4° C.

The platelet pellet was obtained by centrifuging the PRP at room temperature at 1800 rpm (350 g) for 14 minutes. The supernatant was carefully removed using a plastic pasteur pipette, making sure to remove all of the supernatant without disturbing the platelet pellet.

For acid shock permeabilization, 1 ml of incubation buffer per 2 ml PRP was added to a platelet pellet and mixed well (1-2×10⁶ platelets/ml). Incubation buffer is made by adjusting the pH of HEPES-buffered physiological saline to 7.4 using 2 M and 0.2 M NaOH, and adding to 10 ml of this buffer 50 μl hirudin (10 U/ml); 6.25 μl apyrase (20 U/ml); 5.8 mg EDTA; 28 mg ATP; and 0.1 g. trehalose. The mixture was incubated for 10-15 minutes in a 30-35° C. waterbath. After incubation, permeabilization was stopped by the addition of 4 ml (4× the volume of incubation buffer) of stop buffer to each tube of incubation buffer. Stop buffer is made by adjusting the pH of the HEPES-buffered physiological saline to 7.0 using 2 M and 0.2 M NaOH and adding to 10 ml of this buffer 50 μl hirudin (10 U/ml); 6.25 μl apyrase (20 U/ml); and 7.05 mg magnesium sulfate. If the stop buffer contains less than 1% trehalose, it is advantageous to pellet the platelets, e.g., by centrifugation, and resuspend in drying buffer prior to drying.

Membrane permeability was assessed by using the fluorescent dye lucifer yellow which confers fluorescence to permeabilized cells. Cells were analyzed under a Nikon epifluorescent microscope. Another indicator of increased permeability examined was radiolabeled sucrose. Results of these experiments demonstrated that the above acid treatment permeabilized the membranes.

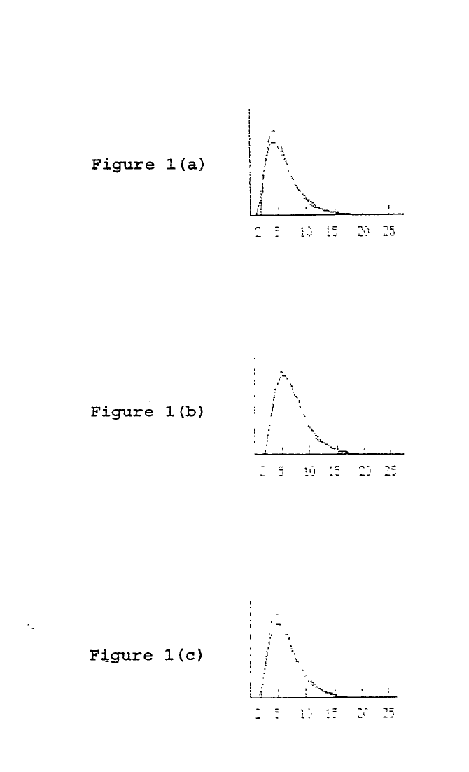

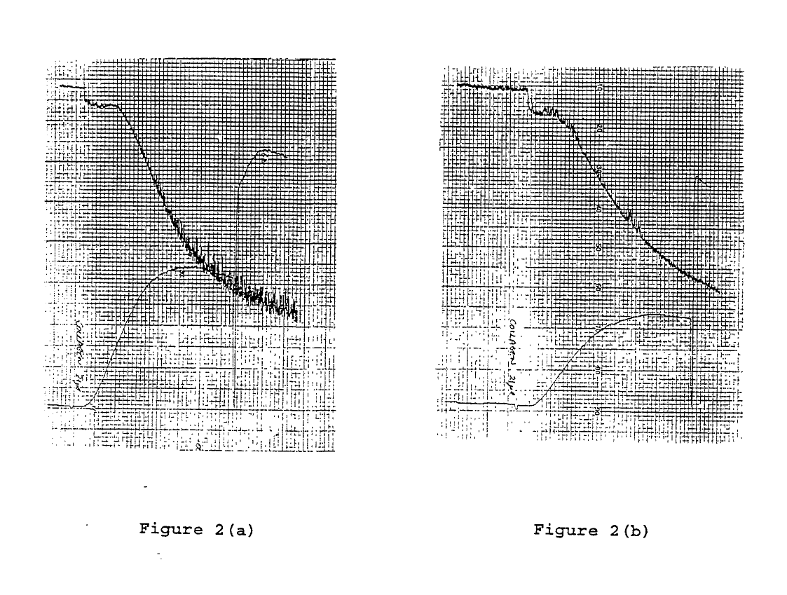

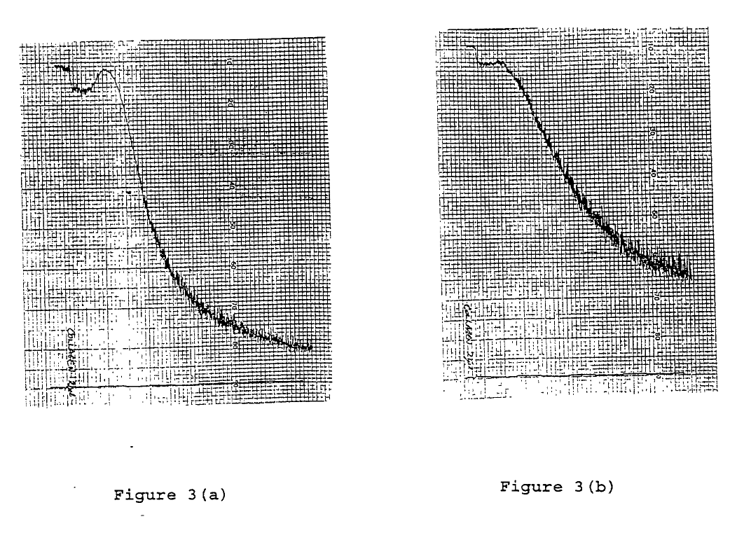

Morphology and functionality of acid permeabilized platelets were analyzed by Coulter® Microdiff 18 hematology analyzer (FIGS. 1(a)-(c), 2(a)-(b), and 3(a)-(b)). The morphology of platelets before and after acid treatment was unchanged (FIGS. 1(a)-(c)). ATP release response of acid-permeabilized platelets was also within normal levels (FIGS. 2(a)-(b)). Platelet aggregation properties were not affected by acid shock permeabilization (FIGS. 3(a)-(b)).

### EXAMPLE 2

### Drying of Platelets Loaded with Trehalose by Acid Shock Permeabilization

Platelets were acid permeabilized as in Example 1. After addition of stop buffer, the mixture was centrifuged at room temperature at 1800 rpm (350 g) for 10 minutes, to pellet the platelets. Supernatant was carefully removed using a plastic pasteur pipette. Platelet pellets were resuspended in drying buffer to a final platelet count of 300-450×10⁹ per liter. Drying buffer is prepared by bringing the pH of HEPES-buffered saline to 7.0 using 2 M and 0.2 M NaOH. To 10 ml of this buffer 50 μl hirudin (loU/ml); 6.25 μl apyrase (20 U/ml); 1 mg magnesium sulphate; 0.1 g trehalose; and 0.1 g. BSA were added. 300 μl of resuspended platelets was carefully pipetted into 3 ml siliconized glass pharmaceutical vials and dried.

The platelets were dried in a modified FTS Systems freeze drier. FTS drying was done by programming the following initial settings into the FTS: shelf temperature, 37° C.; vacuum, 30 Torr. Before starting the run the sample temperature was equilibrated to approximately 35° C. During the run the pressure was gradually decreased so that the sample temperature did not fall below 20° C. The following protocol is adjusted for drying twelve, 3 ml vials containing 300 μl of product:

| | | | approximate |
|---|---|---|---|
| | | shelf | product |
| vacuum | | temperature | temperature |
| (mTorr) | time (min) | (° C.) | (° C.) |
| 30,000 | 4 | 37 | 29 |
| 25,000 | 5 | 60 | 27 |
| 20,000 | 3 | 60 | 24 |
| 15,000 | 11 | 60 | 20 |
| 10,000 | 3 | 60 | 24 |
| | | | (product dry) |
| 2,000 | 2 | 20 | 41 |
| 300 | 8 | 20 | 44 |
| 30 | overnight | 30 | 39 |

For reconstitution, the vials were rehydrated by the addition of 300 μl 20% HSA in saline (BioProducts Laboratories) and gently mixed. Rehydrated platelets were analyzed by Coulter® Microdiff 18 hematology analyzer and light microscopy. Platelet function was assayed by studying aggregation in response to the following agonists:

| | |
|---|---|
| thrombin | 0.77 U/ml (final concentration) |
| collagen | 155 μg/ml (final concentration) |
| ristocetin | 1.16 mg/ml (final concentration) |
| ADP | 10.6 μM (final concentration) |
| epinephrine | 7.7 μmol/l (final concentration) |
| arachidonic | 0.24 mg/ml (final concentration) |
| acid | |

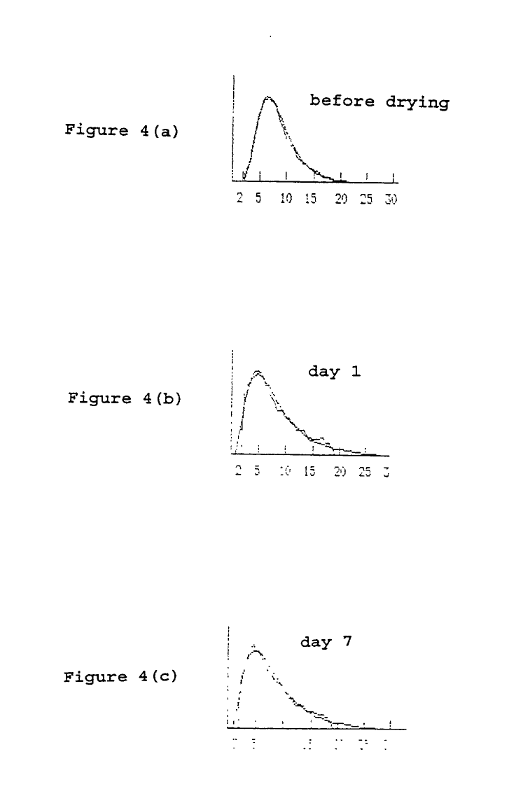

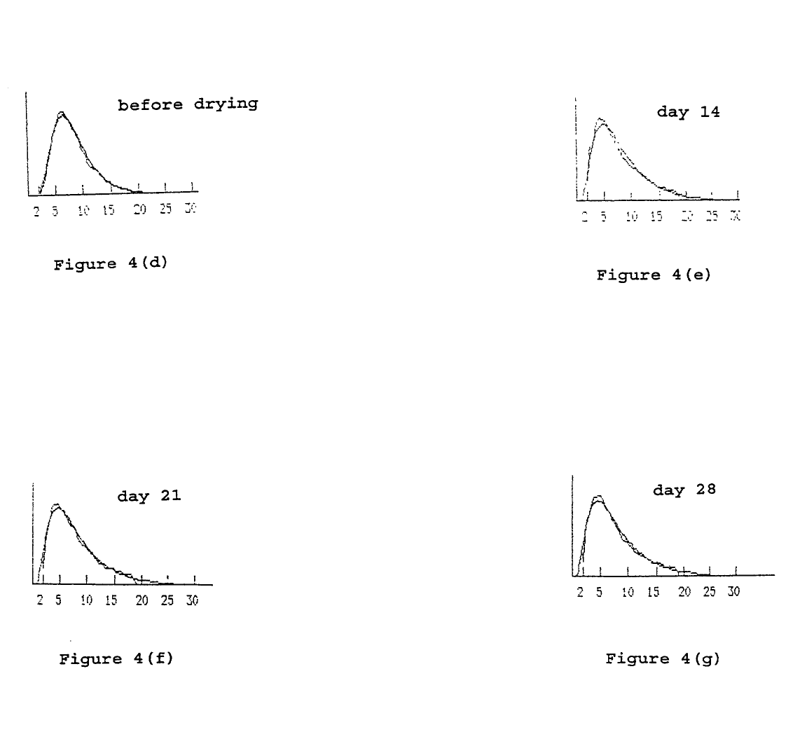

Total numbers and morphology of platelets was not altered after drying and rehydration as assayed using the Coulter® Microdiff 18 hematology analyzer (FIGS. 4(a)-(g)). FIGS. 4(a)-(c) show no change in morphology after 7 days of storage. Platelets maintained normal morphology for as long as 28 days (FIGS. 4(d)-(g)).

The reconstituted platelets displayed normal morphology as analyzed by light microscopy (FIG. 5(a)). When treated with agonists, the reconstituted platelets exhibited normal aggregative function (FIG. 5(b)).

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

We claim:

1. A method for permeabilizing platelets, comprising the step of treating isolated platelets with an acid in an amount and under conditions effective to permeabilize the platelets.

2. The method according to claim 1, wherein the acid is selected from the group consisting of acid-buffered MES, acid-buffered HEPES, and acid-buffered PIPES.

3. The method according to claim 1, wherein the acid is ATP-buffered HEPES.

4. The method according to claim 3, wherein the ATP-buffered HEPES is at pH 4.5.

5. The method according to claim 1, wherein the acid is added to make the pH about 4-5.

6. The method according to claim 1, wherein the acid is added to make the pH about 4.5.

7. A method for loading compounds into platelets, comprising the steps of treating isolated platelets with an acid in an amount and under conditions effective to permeabilize the platelets and incubating the platelets with at least one compound.

8. The method according to claim 7, wherein the compound is a stabilizing agent.

9. The method according to claim 8, wherein the stabilizing agent is trehalose.

10. The method according to claim 8, wherein the stabilizing agent is selected from the group consisting of trehalose, a non-reducing glycoside of polyhydroxy compounds, raffinose, stachyose, melezitose, maltitol, lactitol, and palatinit and its constituent isomers 6-α-D-glucopyranosyl-mannitol and 6-α-D-glucopyranosyl-sorbitol.

11. The method according to claim 8, further comprising adding an amount of an inhibitor of the Maillard reaction effective to inhibit occurrence of the Maillard reaction.

12. The method according to claim 7, wherein the compound is a tracer.

13. The method according to claim 7, wherein the compound is an imaging substance.

14. The method according to claim 13, wherein the imaging substance is a fluorescent tag.

15. The method according to claim 7, wherein the compound is a cryoprotectant.

16. The method according to claim 7, wherein the compound is a bioactive material.

17. The method according to claim 16, wherein the bioactive material is a wound healing factor.

18. The method according to claim 7, wherein the compound is a nucleic acid.

19. The method according to claim 7, further comprising the step of drying the loaded platelets.

20. A method for stabilizing platelets, comprising the steps of:
(a) obtaining platelets;
(b) treating platelets with an acid in an amount and under conditions effective to permeabilize the platelets;
(c) incubating the platelets in the presence of stabilizing agent;
(d) neutralizing the pH; and
(e) drying the platelets.

21. The method according to claim 20, further comprising the steps of:
(a) removing the acid and stabilizing agent from the platelets; and
(b) resuspending the platelets in a buffer comprising a stabilizing agent, wherein steps (a) and (b) are performed after step (d) and before step (e) of claim 20.

22. The method according to claim 20, wherein the acid is selected from the group consisting of acid-buffered MES, acid-buffered HEPES, and acid-buffered PIPES.

23. The method according to claim 20, wherein the acid is ATP-buffered HEPES.

24. The method according to claim 23, wherein the ATP-buffered HEPES is at pH4.5.

25. The method according to claim 23, wherein the ATP-buffered HEPES further comprises effective concentrations of hirudin, apyrase, or indomethacin.

26. The method according to claim 20, wherein the acid is added to make the pH about 4-5.

27. The method according to claim 20, wherein the acid is added to make the pH about 4.5.

28. The method according to claim 20, wherein the platelets are treated with acid for about 5 to 30 minutes.

29. The method according to claim 20, wherein the platelets are treated with acid for about 10-15 minutes.

30. The method according to claim 26, wherein the platelets are treated at a temperature of about 25-40° C.

31. The method according to claim 27, wherein the platelets are treated at a temperature of about 25-40° C.

32. The method according to claim 26, wherein the platelets are treated at a temperature of about 28-37° C.

33. The method according to claim 27, wherein the platelets are treated at a temperature of about 28-37° C.

34. The method according to claim 20, wherein the stabilizing agent is selected from the group consisting of trehalose, a non-reducing glycoside of polyhydroxy compounds, raffinose, stachyose, melezitose, maltitol, lactitol, and palatinit and its constituent isomers 6-α-D-glucopyranosyl-mannitol and 6-α-D-glucopyranosyl-sorbitol.

35. The method according to claim 20, further comprising adding an amount of an inhibitor of the Maillard reaction effective to inhibit occurrence of the Maillard reaction.

36. The method according to claim 24, wherein the stabilizing agent is about 0.1-20% trehalose.

37. The method according to claim 24, wherein the stabilizing agent is about 1-5% trehalose.

38. The method according to claim 24, wherein the stabilizing agent is 1% trehalose.

39. The method according to claim 20, wherein the drying occurs under a vacuum.

40. The method according to claim 39, wherein the vacuum occurs such that the temperature of the sample does not drop below 20° C.

41. The method according to claim 39, wherein the vacuum decreases stepwise such that the temperature of the sample does not drop below 20° C.

42. The method according to claim 20, wherein the drying is by spray drying.

43. The method according to claim 20, wherein the drying is by freeze drying.

44. A method for stabilizing platelets, comprising:
(a) obtaining platelets;
(b) treating platelets with an acid in an amount and under conditions effective to permeabilize the platelets;
(c) incubating the platelets in the presence of about 0.1 to 20% trehalose;
(d) neutralizing the pH; and
(e) drying the platelets.

45. The method of claim 44, wherein the trehalose is about 1%-5%.

46. The method of claim 44, wherein the trehalose is about 1%.

47. A method for stabilizing platelets, comprising:
(a) isolating/enriching platelets from whole blood;
(b) incubating the isolated/enriched platelets for 10-15 minutes at 28-37° C. in ATP-buffered HEPES (pH 4.5), comprising hirudin, apyrase, indomethacin, and trehalose;
(c) adjusting the pH of the incubation mixture to approximately the physiological range;
(d) pelleting the platelets;
(e) resuspending the platelets in HEPES buffered saline, comprising about 1-5% trehalose about 1-2% bovine serum albumin, hirudin, apyrase, and indomethacin;
(f) drying the resuspended platelets under vacuum which is decreased in a stepwise manner while keeping the sample temperature above 20° C.; and
(g) further drying the platelets under vacuum for 16 hours at 30° C.

48. The method according to claim 47, wherein the pH of the incubation mixture in step (c) is adjusted to about 7.0-7.5.

49. A method for reconstitution of dried platelets, comprising resuspending the dried platelets obtained according to claim 19 in a physiologically acceptable buffer.

50. The method according to claim 49, wherein the physiologically acceptable buffer comprises a substance which exhibits high colloidal osmotic pressure.

51. The method according to claim 50, wherein the substance in the physiologically acceptable buffer is selected from the group consisting of HSA, PEG, and HES.

52. The method according to claim 49, wherein the physiologically acceptable buffer is 20% HSA in saline.

53. A method for reconstitution of dried platelets, comprising resuspending the dried platelets obtained according to claim 20 in a physiologically acceptable buffer.

54. The method according to claim 53, wherein the physiologically acceptable buffer comprises a substance which exhibits high colloidal osmotic pressure.

55. The method according to claim 54, wherein the substance in the physiologically acceptable buffer is selected from the group consisting of HSA, PEG, and HES.

56. The method according to claim 53, wherein the physiologically acceptable buffer is 20% HSA in saline.

57. A method for reconstitution of dried platelets comprising resuspending the dried platelets obtained according to claim 44 in a physiologically acceptable buffer.

58. The method according to claim 57, wherein the physiologically acceptable buffer comprises a substance which exhibits high colloidal osmotic pressure.

59. The method according to claim 58, wherein the substance in the physiologically acceptable buffer is selected from the group consisting of HSA, PEG, and HES.

60. The method according to claim 57, wherein the physiologically acceptable buffer is 20% HSA in saline.

61. A method for reconstitution of dried platelets, comprising resuspending the dried platelets obtained according to claim 47 in a physiologically acceptable buffer.

62. The method according to claim 61, wherein the physiologically acceptable buffer comprises a substance which exhibits high colloidal osmotic pressure.

63. The method according to claim 62, wherein the substance in the physiologically acceptable buffer is selected from the group consisting of HSA, PEG, and HES.

64. The method according to claim 61, wherein the physiologically acceptable buffer is 20% HSA in saline.

65. A composition comprising dried, storage-stable platelets obtained according to the method of claim 19.

66. A composition comprising dried, storage-stable platelets obtained according to the method of claim 20.

67. A composition comprising dried, storage-stable platelets obtained according to the method of claim 44.

68. A composition comprising dried, storage-stable platelets obtained according to the method of claim 47.

69. A composition comprising platelets obtained according to the method of claim 1.

70. A composition comprising platelets obtained according to the method of claim 7.

71. A composition of reconstituted, storage-stable platelets comprising the platelets obtained according to the method of claim 49 in a physiologically acceptable buffer.

72. The composition according to claim 71, wherein the physiologically acceptable buffer comprises a substance which exhibits high colloidal osmotic pressure.

73. The composition according to claim 72, wherein the substance in the physiologically acceptable buffer is selected from the group consisting of HSA, PEG, and HES.

74. The composition according to claim 71, wherein the physiologically acceptable buffer is 20% HSA in saline.

75. A composition of reconstituted, storage-stable platelets comprising the platelets obtained according to the method of claim 53 in a physiologically acceptable buffer.

76. The composition according to claim 75, wherein the physiologically acceptable buffer comprises a substance which exhibits high colloidal osmotic pressure.

77. The composition according to claim 76, wherein the substance in the physiologically acceptable buffer is selected from the group consisting of HSA, PEG, and HES.

78. The composition according to claim 75, wherein the physiologically acceptable buffer is 20% HSA in saline.

79. A composition of reconstituted, storage-stable platelets comprising the platelets obtained according to the method of claim 57 in a physiologically acceptable buffer.

80. The composition according to claim 79, wherein the physiologically acceptable buffer comprises a substance which exhibits high colloidal osmotic pressure.

81. The composition according to claim 80, wherein the substance in the physiologically acceptable buffer is selected from the group consisting of HSA, PEG, and HES.

82. The composition according to claim 79, wherein the physiologically acceptable buffer is 20% HSA in saline.

83. A composition of reconstituted, storage-stable platelets comprising the platelets obtained according to the method of claim 61 in a physiologically acceptable buffer.

84. The composition according to claim 83, wherein the physiologically acceptable buffer comprises a substance which exhibits high colloidal osmotic pressure.

85. The composition according to claim 84, wherein the substance in the physiologically acceptable buffer is selected from the group consisting of HSA, PEG, and HES.

86. The composition according to claim 83, wherein the physiologically acceptable buffer is 20% HSA in saline.

87. The composition according to claim 65, further comprising a pharmaceutically acceptable vehicle or excipient.

88. The composition according to claim 66, further comprising a pharmaceutically acceptable vehicle or excipient.

89. The composition according to claim 67, further comprising a pharmaceutically acceptable vehicle or excipient.

90. The composition according to claim 68, further comprising a pharmaceutically acceptable vehicle or excipient.

91. The composition according to claim 69, further comprising a pharmaceutically acceptable vehicle or excipient.

92. The composition according to claim 70, further comprising a pharmaceutically acceptable vehicle or excipient.

93. The composition according to claim 71, further comprising a pharmaceutically acceptable vehicle or excipient.

94. The composition according to claim 75, further comprising a pharmaceutically acceptable vehicle or excipient.

95. The composition according to claim 79, further comprising a pharmaceutically acceptable vehicle or excipient.

96. The composition according to claim 83, further comprising a pharmaceutically acceptable vehicle or excipient.

97. A method for delivering therapeutic agents, comprising:
(a) obtaining a sample enriched in platelets;
(b) treating the platelets with an acid in an amount and under conditions effective to permeabilize the platelets;
(c) incubating the permeabilized platelets with a stabilizing agent;
(d) incubating the platelets with a therapeutic agent; and
(e) administering the platelets.

98. The method according to claim 97, further comprising drying and recovering the platelets.

99. The method according to claim 97, wherein the therapeutic agent is a wound healing factor.

100. A method for producing platelets suitable for purification of platelet factors, comprising:
(a) treating platelets with an acid in an amount and under conditions effective to induce permeabilization;
(b) incubating the permeabilized platelets with a stabilizing agent; and
(c) drying and recovering the platelets.

101. The method according to claim 100, wherein the platelets factor is PDGF.

102. The method according to claim 100, wherein the platelets factor is vWF.

103. A composition comprising platelets obtained according to the method of claim 100.
